(19) United States
(12) Patent Application Publication
(10) Pub. No.: US 2010/0262198 A1
(43) Pub. Date: Oct. 14, 2010

(54) Instrument set for inserting a stabilization system into the spinal column of a body

(76) Inventors: Reto Braunschweiler, Neftenbach (CH); Simon Fischli, Ontario (CA)

Correspondence Address: JACOBSON HOLMAN PLLC, 400 SEVENTH STREET N.W., SUITE 600, WASHINGTON, DC, 20004, US

(21) Appl. No.: 12/659,946

(22) Filed: Mar. 25, 2010

(30) Foreign Application Priority Data
Mar. 26, 2009 (EP) 09156346.0

(51) Int. Cl. A61B 17/56

(52) U.S. Cl. 606/86 A

(57) ABSTRACT

An instrument set for inserting a stabilization system into a vertebral column of a body incorporates means for inserting anchoring elements (1) into the respective vertebral bodies (2), means for inserting a stabilizing rod (24) into the fixed anchoring elements (1) and means (38) for fitting and bracing of the locking devices onto the anchoring elements. The means for inserting the anchoring elements (1) have a head and a shaft. The head is so constructed that it can be inserted into the U-shaped head part of the anchoring element (1). The shaft is equipped with first guide means, along which pliers (14) equipped with second guide means (20) are guided onto the head part of the anchoring element (1) and coupled with it. The means for inserting the rod and the means (38) for fitting and bracing the locking devices are equipped with further guide means (42), which interact with the second guide means (20) of the pliers. In this way, accurate fixation of such a stabilization system onto the vertebral column of a body is guaranteed.

The present invention relates to an instrument set for inserting a stabilization system, comprising anchoring elements, each having a screw holder and a U-shaped head part, into which head part a rod can be inserted for stabilization and be locked and latched by means of a locking device, into the spinal column of a body, comprising means for inserting the anchoring elements into the respective bone or vertebral bodies, means for inserting the stabilization rod into the inserted anchoring elements, and means for fitting and bracing the locking devices to the anchoring elements.

Anchoring elements, into which a rod can be inserted, which rod is then held into the anchoring element by a locking device fitted to the anchoring element, are known in the art in diverse ways. Stabilization systems consisting of anchoring elements, locking devices, and rod serve to stabilize patient's spinal column, which spinal columns show extensive damage. To do so, one anchoring element is inserted respectively into a number of vertebral bodies, for example by screw connection, a rod is inserted into the head parts of these anchoring elements, and this rod is then connected respectively to the corresponding anchoring element, the locking devices are being used for this connection. Two different forms of stabilization can be achieved during this process, using a rigid rod achieves a stiffening of the affected vertebral bodies, using an elastic rod achieves a supporting stabilization of the vertebral bodies with a certain flexibility between the individual vertebral bodies.

A known type of anchoring elements are pedicle screws, for example. A pedicle screw for a stabilization system with locking device and rod can be found in European Patent application No. 07150489.8, for example. The pedicle screws are screwed into the respectively prepared vertebral bodies by means of a corresponding tool, the rod is inserted into the U-shaped head parts of these screws, a locking device is fitted onto each screw, the latching part is set to the locked position, the clamping bolt is tightened. For insertion of this stabilization system into the spinal column of a body, instruments are used as assistive means. It is of critical relevance during this process that the stabilization system inserted into the vertebral column is inserted absolutely correctly to exclude that the stabilization system can change at a later point in time due to incorrect handling, for example by moving the rod in the pedicle screws, which could have unforeseeable consequences.

Object of the present invention is thus to create an instrument set for inserting a stabilization system into the spinal column of a body, which enables that particularly the insertion of the rod into the anchoring elements, the fitting of the locking device onto the anchoring elements and the latching of the locking device to the anchoring elements, and the bracing of the rod can be implemented in a simple and reliable way.

This object is achieved according to the invention in that the means for inserting the anchoring element have a head and a shaft, in that the head is formed such that it can be inserted into the U-shaped head part of the anchoring element, in that the shaft is equipped with first guide means, along which pliers equipped with second guide means can be fitted to and coupled with the anchoring element, and in that the means for inserting the rod and the means for fitting and bracing the locking device are equipped with further guide means, which interact with the second guide means of the pliers.

With such an instrument set for inserting a stabilization system into a spinal column of a body, each individual instrument that is used for insertion of this stabilization system is always guided with respect to the head part, the placing of the rod and fitting of the locking device on the anchoring element as well as the latching and bracing of the rod is implemented exclusively by means of distinctively guided and positioned instruments, thereby practically excluding incongruous manipulations with the mentioned serious consequences.

The first guide means are advantageously mounted to a guide sleeve, which guide sleeve is slidable onto the shaft of the screw-in means. This achieves a simple embodiment of the means for insertion of the anchoring elements.

The pliers are advantageously furnished with jaws, which grip the two branches of the U-shaped head part on the outer sides and which are furnished with cams, which engage into recesses mounted on both branches respectively, and by which the pliers are coupled to the anchoring element. This achieves a secure mount of the pliers to the anchoring element on the one hand, and on the other hand, the area of the anchoring element, which has to accept the rod and the locking device, is freely accessible.

The jaws of the pliers are advantageously disposed offset in the direction of the pivot axis, thereby keeping free the access to the anchoring element in axial direction.

The pliers are advantageously lockable in the coupled position with the anchoring element by means of arresting means, which ensures that the connection between anchoring element and the pliers fitted thereon can not be loosened accidentally.

Another advantageous embodiment of the invention is that the means for inserting the rod into the anchoring element are formed of one bar with a press-in part mounted to the front end and a handle mounted on the rear end, and that the bar is furnished with third guide means, which interact with the second guide means of the pliers. Aside from the simple handling, this also ensures that the means for inserting the rod are always in an exactly guided position with respect to the anchoring element.

The means for fitting and bracing of the locking device are advantageously formed from one sleeve, the front end of which has a seating part for accepting the locking device, and which sleeve is furnished with four guide means that interact with the second guide means of the pliers. This also ensures that the locking device can be exactly positioned onto the anchoring element with the help of the sleeve.

The locking device advantageously consists of an insertion part, a latching part, and a clamping bolt, which locking device can be optimally fitted to, latched and clamped onto the anchoring element.

The seating part is advantageously furnished with slot-shaped recesses in which the latched part of the locking device can be screwed and which hold the locking device in the seating part, thereby preventing an unintended loss of the locking device from the seating part.

Another advantageous embodiment of the invention is that a rotating element is inserted into the sleeve, the front end of which is furnished with projections, which project over the latching part in the inserted position of the locking device into the seating part and thus cause the latching part to be rotatable with respect to the insertion part by rotating the rotating element, thereby enabling positional accuracy and easy latching of the locking device with the anchoring element.

To be able to facilitate easy handling of the sleeve and the rotating element, the sleeve is furnished with a handle on its rear end, while the rotating element is furnished with a rotary knob on the rear end that protrudes over the sleeve.

Another advantageous embodiment of the invention is that the rotating element is furnished with a continuous longitudinal borehole and that a screwdriver, with which the clamping bolt can be rotated, can be inserted into the continuous longitudinal borehole. This also ensures that the insertion of the screwdriver and the tensioning of the clamping screw of the locking device can be implemented optimally.

One embodiment of the invention will be explained in more detail below with respect to the accompanying drawings, whereas the anchoring element in the embodiment example is furnished as a pedicle screw. Of course, other embodiments of anchoring elements are conceivable, as well.

The drawings show:

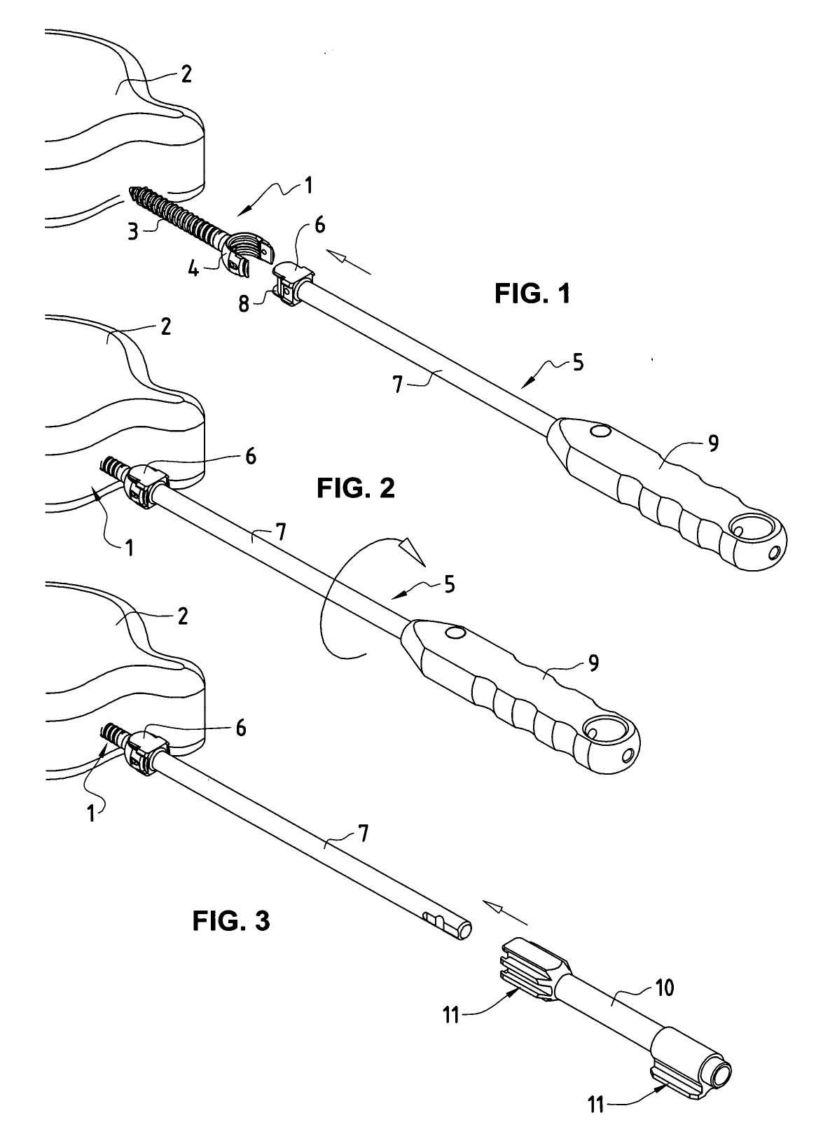

FIG. 1 in spatial illustration, the means for screwing the pedicle screw into a vertebral body;

FIG. 2 in spatial illustration, the means for screwing the pedicle screw during the process of tightening;

FIG. 3 in spatial illustration, the shaft of the means for tightening the pedicle screw and the guide sleeve, which can be fitted onto the shaft;

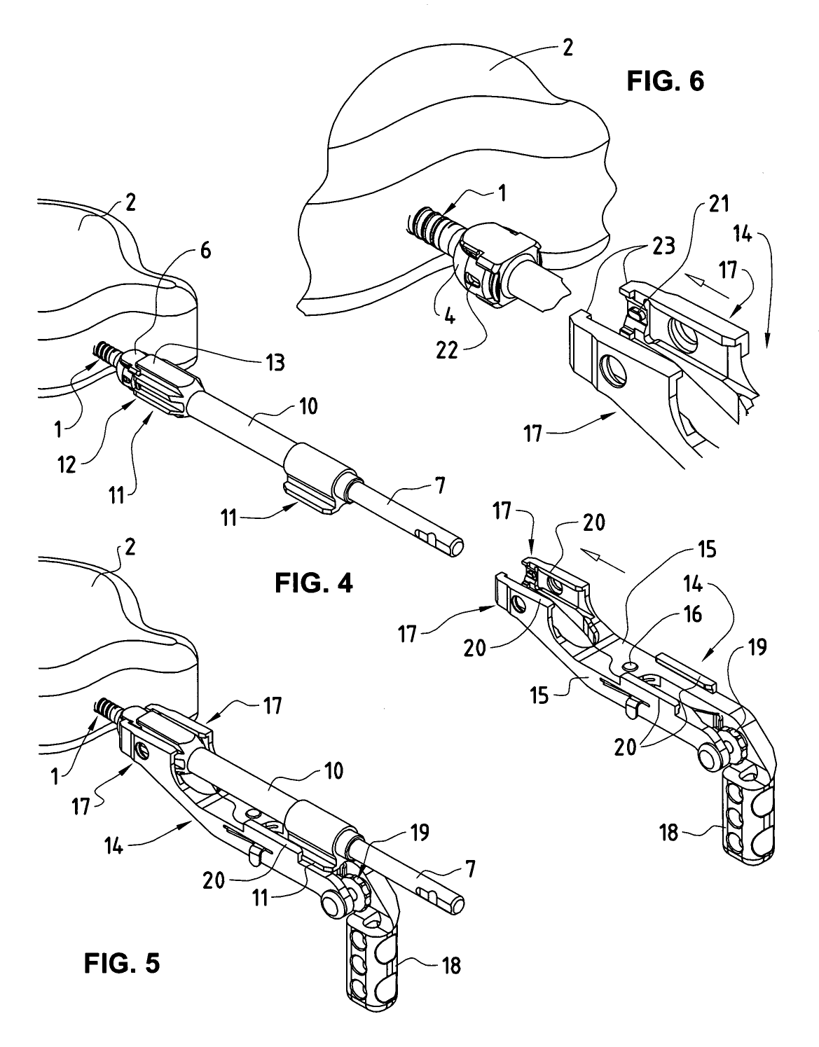

FIG. 4 in spatial illustration the shaft with fitted guide sleeve and the pliers, which can be slid onto the guide sleeve;

FIG. 5 in spatial illustration, the pliers fitted onto the guide sleeve in the coupled in position with the pedicle screw;

FIG. 6 a spatially enlarged illustration of the pliers' jaws and the pedicle screw with fitted shaft;

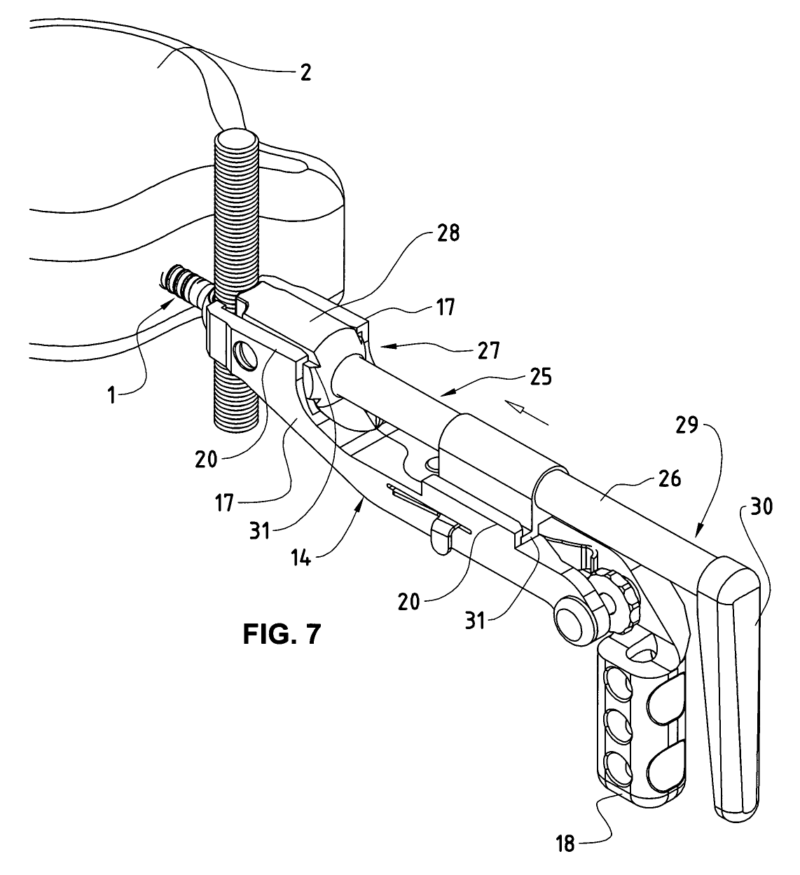

FIG. 7 in spatial illustration, the means for inserting the rod into the pedicle screw in the position of being inserted into the pliers;

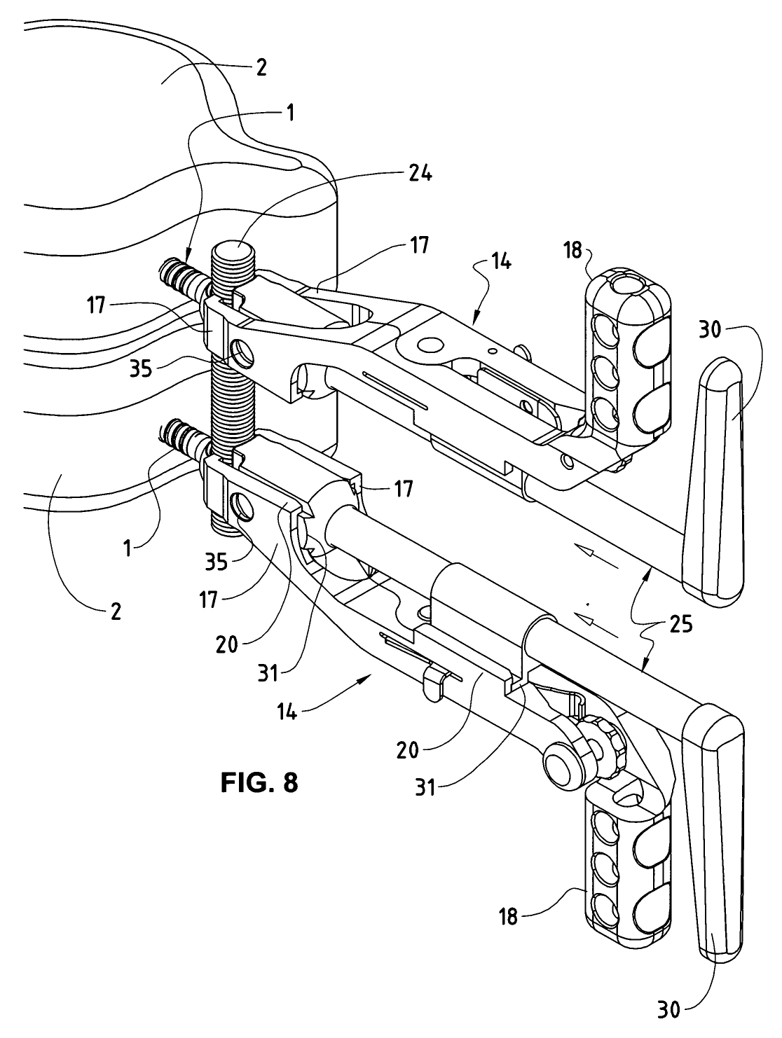

FIG. 8 in spatial illustration, two vertebral bodies with inserted pedicle screw, each with fitted pliers and inserted means for insertion of the rod into the pedicle screws;

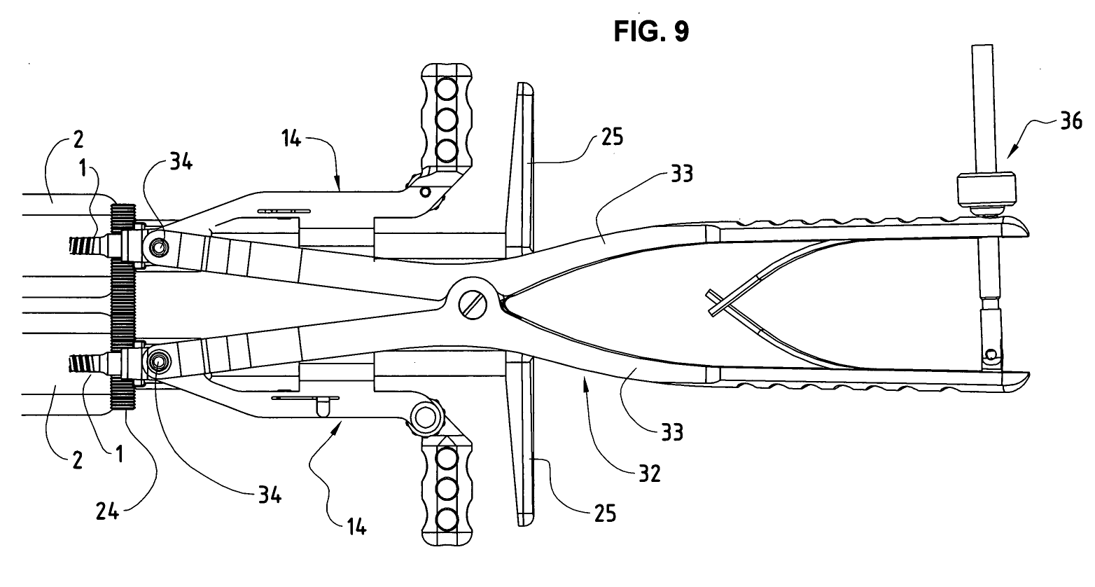

FIG. 9 a view of an instrument for distraction of two adjacent vertebral bodies;

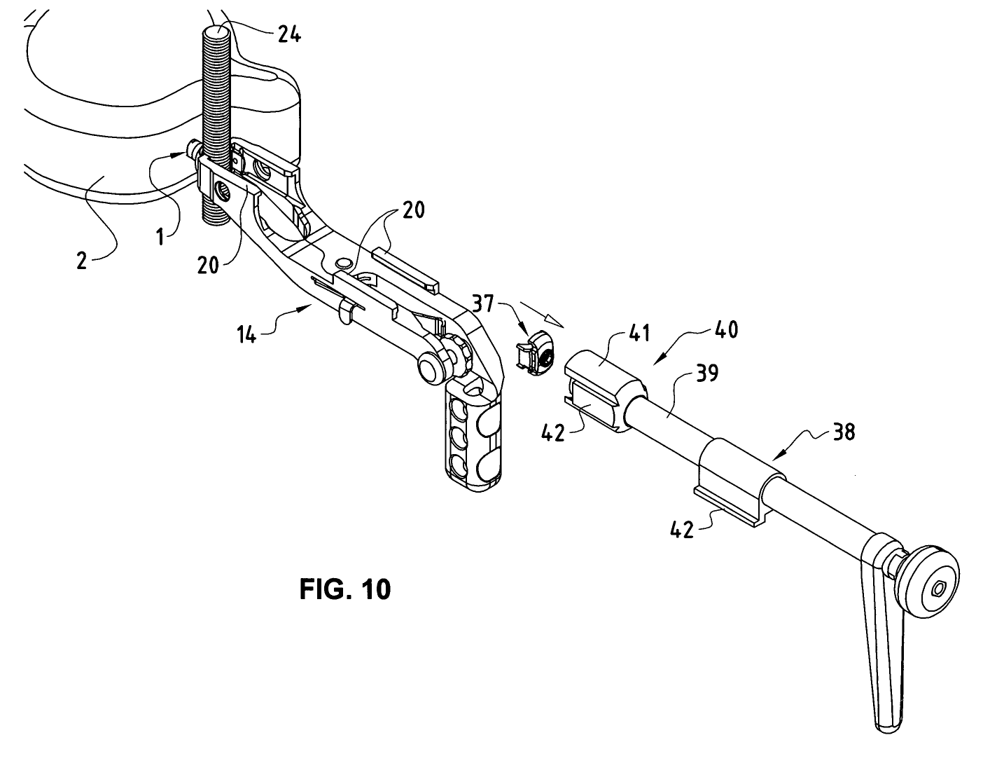

FIG. 10 in spatial illustration, the pliers attached onto the pedicle screw, the locking device and the means to fit and brace the locking device;

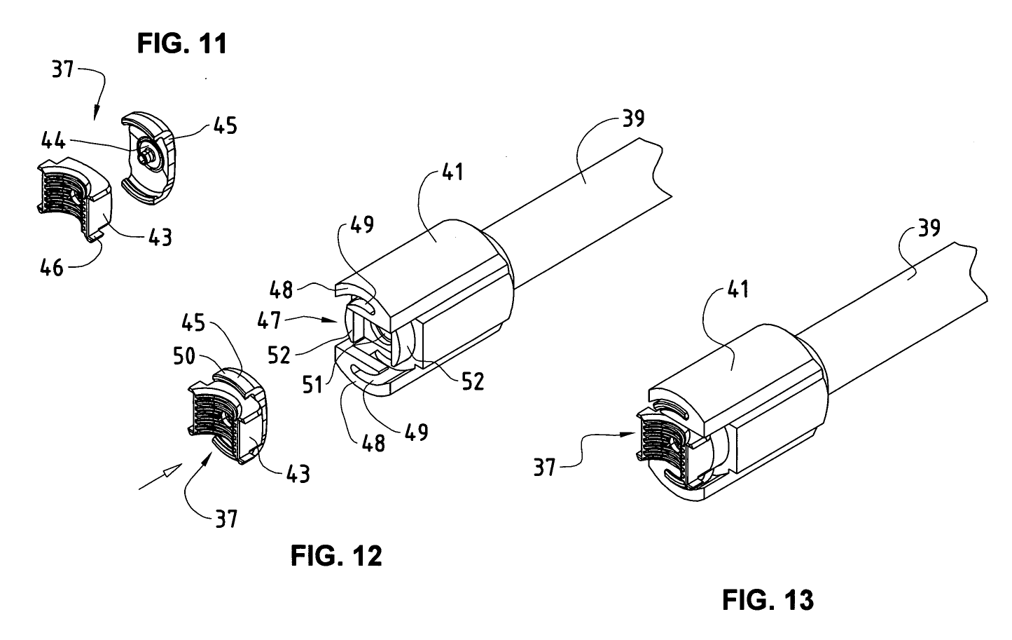

FIG. 11 in spatial illustration, the locking device;

FIG. 12 in spatial illustration, the seating part of the means to fit and clamp the locking device and the locking device that is insertable therein;

FIG. 13 in spatial illustration, the seating part with the locking device inserted therein;

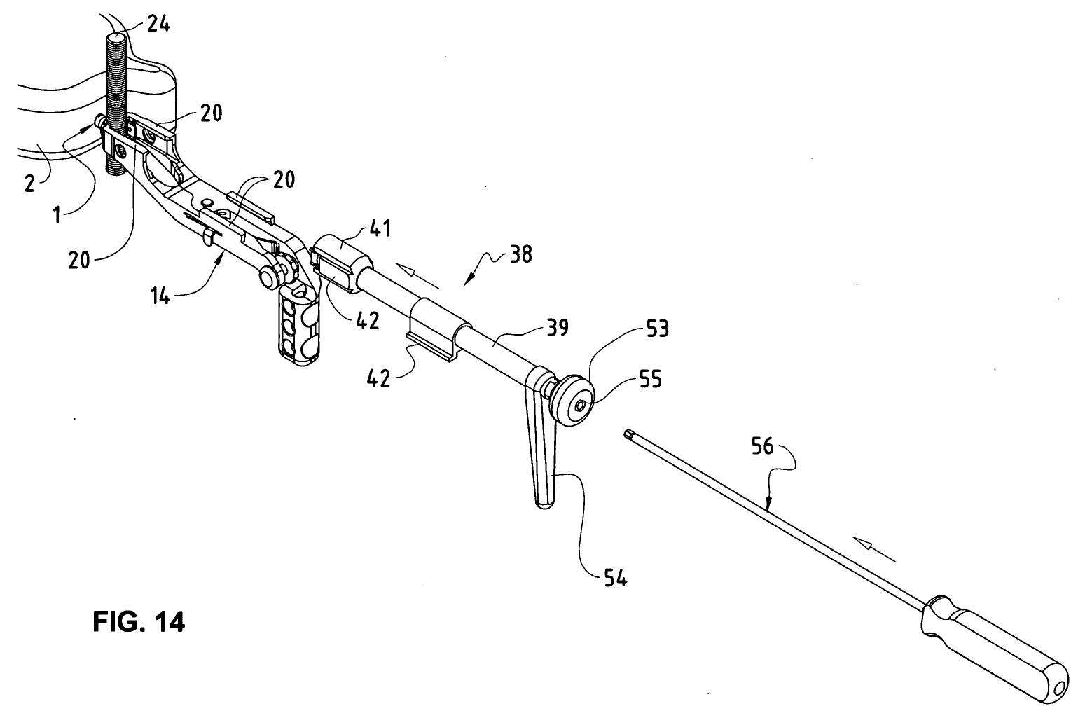

FIG. 14 in spatial illustration, the pliers fitted onto the pedicle screw, the means to fit and clamp the locking device and the screwdriver that can be inserted into the means for fitting and clamping the locking device;

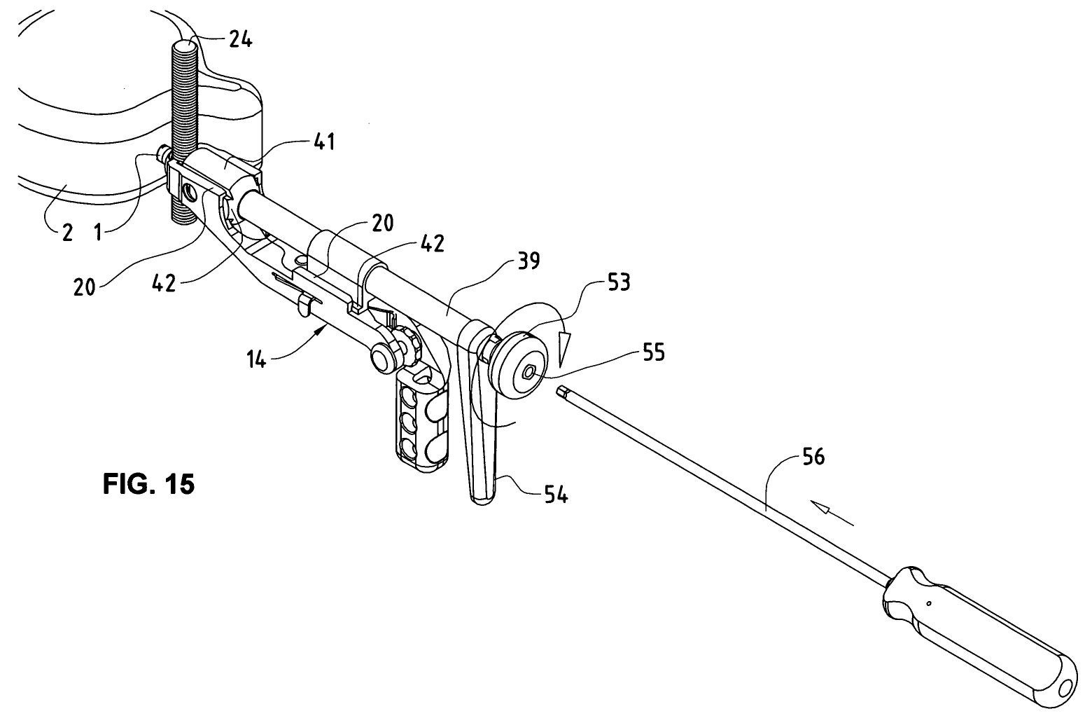

FIG. 15 in spatial illustration, the inserted means into the pliers for fitting onto and clamping of the locking device;

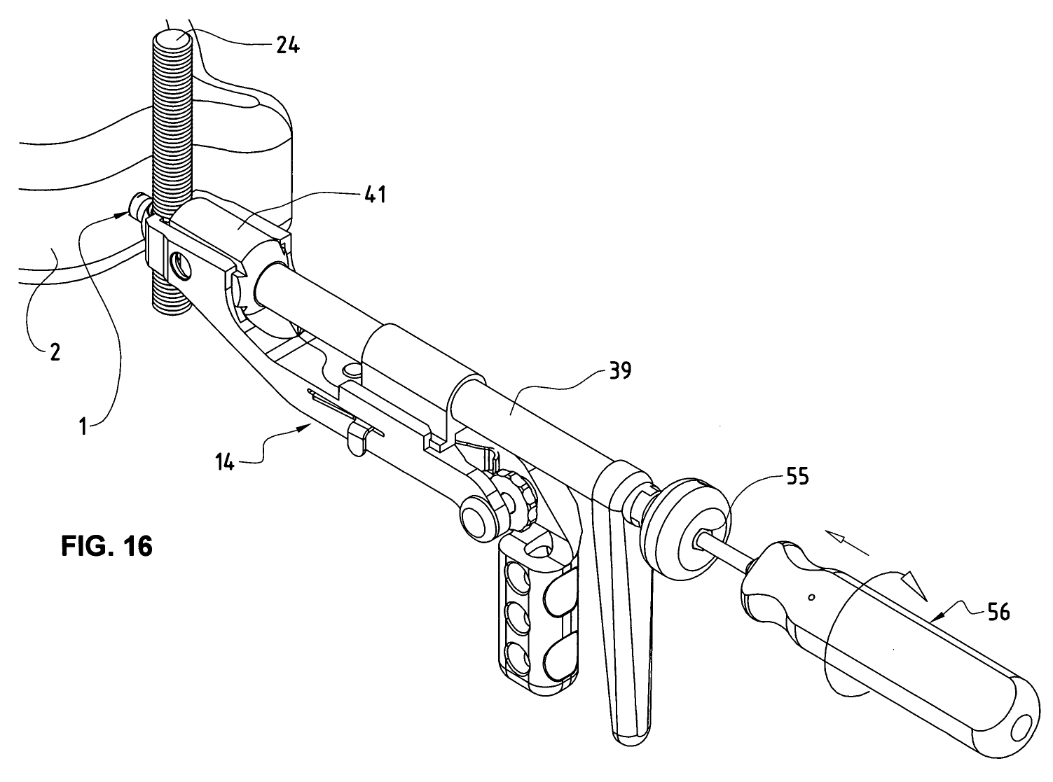

FIG. 16 in spatial illustration, the screwdriver, which has been inserted into the means for fitting onto and clamping of the locking device, for tightening of the clamping screw;

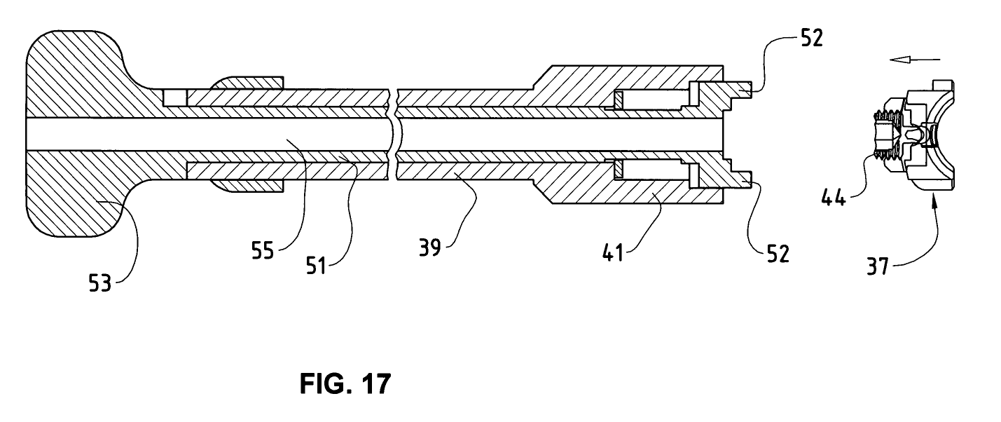

FIG. 17 a cross-sectional view of the means for fitting and clamping of the locking device; and

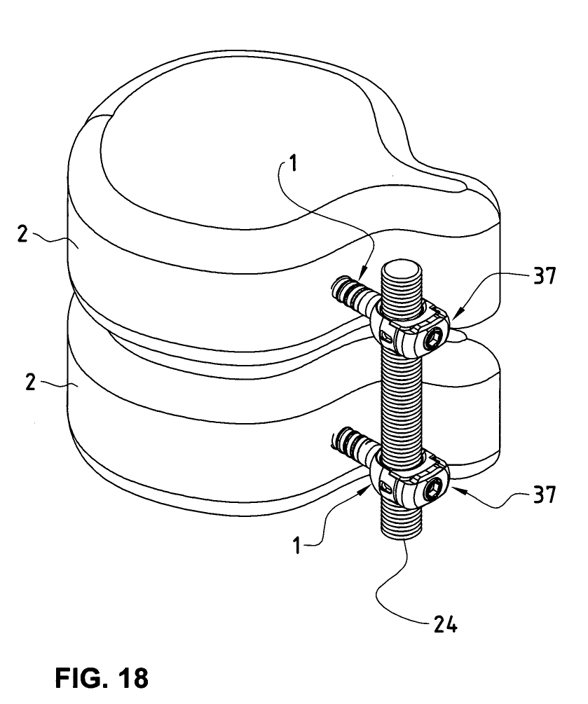

FIG. 18 in spatial illustration, the stabilization system inserted into the vertebral bodies.

FIG. 1 shows pedicle screw 1, which is to be screwed into vertebral body 2. It is known in the art that pedicle screw 1 is furnished with a screw-in part 3 and a U-shaped head part 4. For tightening of pedicle screw 1, means 5 are used that consist of head 6 and a shaft 7. Head 6 is furnished in a way that it can be suitably inserted into the U-shaped head part 4 of the pedicle screw 1. Head 6 is particularly fitted with guide ribs 8, which align pedicle screw 1 coaxially to the shaft 7 and hold head 6, pedicle screw 1 being attached onto the means for tightening 5. At the end region averted from head 6, a handle 9 is attached onto shaft 7 for better handling of the means for tightening 5.

As can be seen from FIG. 2, the pedicle screws 1 are screwed into the respectively prepared vertebral bodies 2 with these means for tightening 5.

As seen from FIG. 3, handle 9 has been loosened and detached from shaft 7, with which it had been connected via latch means known in the art. Shaft 7 remains connected to the U-shaped head part 4 of pedicle screw 1. A guide sleeve 10 can be fitted onto shaft 7, which is furnished with first guide means 11.

FIG. 4 shows guide sleeve 10, which is attached onto shaft 7. Guide sleeve 10 is furnished with two protruding tongues 13 at the front-end region 12, the head 6 fitting exactly between the two tongues 13. This defines the angular position of guide sleeve 10 with respect to head 6 and thus pedicle screw 1. To show that guide sleeve 10 has taken the correct position with respect to shaft 7 or head 6 and thus pedicle screw 1, markings known in the art (not shown) can be applied to guide sleeve 10 and shaft 7, thereby enabling visual control of the exact position of guide sleeve 10 with respect to pedicle screw 1.

Pliers 14 can be fitted onto these first guide means 11 of guide sleeve 10, as described in detail below. These pliers 14 comprise two levers 15, which can be swiveled against each other via a pivot axis 16. The front ends of levers 15 are furnished with a respective jaw 17, the rear end of one lever 15 is fitted with a handle 18, arresting means 19 known in the art are mounted between the two rear ends of lever 15. The pliers 14 are furnished with second guide means 20, pliers 14 thus can be slid—guided via the second guide means 20 over the first guide sleeve 11 of guide sleeve 10—onto this guide sleeve 10; FIG. 5 shows the slid-on position.

As can be seen from the enlarged view in FIG. 6, jaws 17 of pliers 14 are formed such that these can grip the two branches of the U-shaped head part 4 of pedicle screw 1 on the outer sides. Jaws 17 of pliers 14 are furnished with cams 21, which can engage the recesses 22 attached to the respective branches of the U-shaped head part 4. Additionally, jaws 17 of pliers 14 are furnished with protrusions 23, which encompass the branches of the U-shaped head part 4 such that together with cams 21, which engage the recesses 22, a turning and tilting of the pliers with respect to the pedicle screw 1 is rendered impossible.

In the fully slid-on position of pliers 14 onto guide sleeve 10 and the U-shaped head part 4 of pedicle screw 1, as shown in FIG. 5, the arresting means 19 can be actuated, pliers 14 thereby being firmly connected to pedicle screw 1.

As can be seen from FIGS. 4 to 6, jaws 17 of pliers 14 are positioned offset in the direction of pivot axis with respect to the swivel axis 16 of pliers 14. That means that pliers 14—in the attached position onto pedicle screw 1 and coupled in axial direction against pedicle screw 1—grants a clearance so that access onto pedicle screw 1 through and between jaws 17 in axial direction can be completely ensured. The correct position of pliers 14 with respect to guide sleeve 10 and corresponding to pedicle screw 1 can visually be shown via markings known in the art (not shown) attached onto guide sleeve 10 and pliers 14.

After pliers 14 are coupled to pedicle screw 1 in the manner described above and shown in FIG. 5, guide sleeve 10 and shaft 7 can be withdrawn from pedicle screw 1 in axial direction, guided by guide means 11, which interact with second guide means 20.

As can be seen in FIG. 7, rod 24, which can be inserted into pedicle screw 1, can be placed into the clearance between the two jaws 17 of pliers 14 of pedicle screw 1, and rod 24 can thereafter be pressed into the U-shaped head part 4 of pedicle screw 1 via means 25 for inserting rod 24 into pedicle screw 1. These means 25 for inserting the rod 24 are formed from a bar 26 on whose distal end 27 a press-in part 28 and on whose proximal end 29 a handle 30 is attached. The bar 26 is provided with third guide means 31, and these third guide means 31 are inserted into the second guide means 20 of the pliers 14 when the rod 24 is inserted into the U-shaped head part 4 of the pedicle screw 1, so that the means 25 for inserting the rod into the pedicle screw are likewise precisely guided during the insertion action in regard to pedicle screw 1. In order to insert the rod 24 into the U-shaped head part 4 of the pedicle screw, the handle 18 of the pliers 14 and the handle 30 of the means 25 for inserting the rod 24 can be pressed against one another in order to exert as good as no force at all on the vertebral body 2 into which the corresponding pedicle screw 1 has been screwed.

FIG. 8 shows two vertebral bodies 2 to be stabilized. One pedicle screw 1 has been screwed into each of these vertebral bodies 2 as per the procedure described above. Pliers 14 have been fit to each of the pedicle screws 1 and coupled to the pedicle screw 1. The rod 24, which is to be inserted into the pedicle screws 1, has been placed into the clearance between the jaws 17 of the two pliers 14, the rod 24 thereafter being pressed into the pedicle screws 1 by the means 25 for inserting the rod 24 into the pedicle screws 1, since the two means 25 for inserting the rod 24, guided by the second guide means 20 of the pliers 14 and the third guide means 31 of the means 25 for inserting the rod 24, are pressed against the pedicle screws 1.

Before the rod 24 is inserted into the pedicle screws 1, as described in FIG. 8, both vertebral bodies 2 can be distracted, i.e., the desired adjustment of the vertebral positions is carried out. For this purpose a distraction tool 32, formed from two articulated levers 33 connected together, can be applied as shown in FIG. 9. At the distal end of these levers 33, screw-head shaped elevations 34 are fixed, that can be inserted into corresponding drill holes 35 (FIG. 8) which are provided laterally on the jaws 17 of the pliers 14. At the proximal end of the levers 33, a known adjusting device 36 is affixed, with which the distal ends of the levers 33 can be pressed together or spread apart. With this, the desired position of the vertebrae can be achieved. The configuration of the screw-head shaped elevations 34 and the drill holes 35 allows the distraction tool 32 to be latched into the pliers 14, so that undesirable rotation of the pliers 14 and the corresponding pedicle screws 1 is prevented. Once the vertebral bodies 2 have been brought into the desired position, the required length of the rod 24 that is to be inserted into the pedicle screws 1 can be determined by measurement, the rod to be inserted can be cut to the appropriate length and then inserted into the pedicle screws 1, as already described in relation to FIG. 8.

In the embodiment illustrated here, an elastic rod is inserted, which consists of a material based on polyurethane, and whose surface has ribs and grooves. The seating part of the U-shaped head part 4 is likewise provided with corresponding ribs and grooves. Once inserted in the pedicle screws 1, the rod 24 is thus locked into place with positive fit so that displacement of the pedicle screws 1 along the longitudinal axis of the rod is prevented. Other materials and surface textures can of course be used for the rod and the seating region of the U-shaped head part of the pedicle screw.

After the rod 24 is inserted into the pedicle screws 1, the means 25 for inserting the rod can be withdrawn from the pliers 14 along the second guide means 20.

The locking device 37 can now be applied to the pedicle screws 1 into which the rod 24 has been inserted, using means 38 for fitting and bracing as can be seen in FIG. 10. The means 38 for fitting and bracing the locking device 37 to the pedicle screw 1 are formed from a sleeve 39, on whose distal end 40 a seating part 41 is fixed for reception of the locking device 37. This sleeve 39 is equipped with fourth guide means 42, which can interface with the second guide means 20 of the pliers 14, as described below.

FIG. 11 shows the construction of a known locking device 37, as described in detail in the European patent application with application number 07150489.8. This locking device comprises an insertion part 43, a clamping bolt 44 and a latching part 45. The insertion part 43 can be applied between the branches of the U-shaped head part 4; the latching part 45 is then rotated about the clamping bolt 44 relative to the insertion part 43, so that the cams 46 engage in a known manner with corresponding slots on the pedicle screw 1. The clamping bolt 44 is then tightened, the insertion part 43 is pressed against the rod inserted into the pedicle screw 1, and the rod is thus held optimally in the pedicle screw.

The region of the seating part 41, which serves to receive the locking device 37, can be seen in FIG. 12. The pre-assembled locking device 37 is brought into the locked position, meaning that the latching part 45 is rotated relative to the insertion part 43 by 90° from the position illustrated in FIG. 12. The locking device 37 is thus placed in the locked state. The locking device 37 can be inserted into the slot-shaped socket 47 of the seating part 41. Each of the two jaws 48, which form the slot-shaped socket 47, is provided with a slot-shaped recess 49. In its position when inserted into the slot-shaped recess 47, the latching part 45 can be rotated back again, the edges 50 of the latching part 45 then enter the slot-shaped recesses 49 of the seating part 41; the locking device 37 is thus firmly held in the seating part 41 of the means 38 for fitting and bracing of the locking device, as can be seen in FIG. 13.

In order to rotate the latching part 45 of the locking device 37 in the inserted state into the seating part 41 of the means 38 for fitting and bracing, a rotating element 51 is inserted into the sleeve 39 as can be seen especially in FIG. 17. The distal end of this rotating element 51 is provided with projections 52, which protrude over the latching part 45 when the locking device 37 is inserted into the seating part 41. By turning this rotating element 51, the latching part 45 can be rotated relative to the insertion part 43 of the locking device 37. These projections 52 fixed to the rotating element 51 can also be seen in FIG. 12.

The means 38 for fitting and bracing of the locking device 37 to the pedicle screw 1 are now guided axially toward the pedicle screw 1, so that the fourth guide means 42 enter the second guide means 20, as can be seen in FIGS. 14 and 15. For easier handling, a handle 53 is attached at the proximal end of the sleeve 39. Due to the accurate guidance of the sleeve 39 relative to the pliers 14, the locking device 37 held in the seating part 41 is accurately placed on the U-shaped head part 4 of the pedicle screw 1. The insertion part 43 of the locking device 37 is introduced between the two branches of the U-shaped head part 4 of the pedicle screw 1; the latching part 45 can be brought into the locked position by turning the rotating element 51 relative to the pedicle screw 1. The rotating element 51 is provided with a handle 53 at its proximal end for this purpose. With markings (not shown) on the sleeve 39 and on the pliers 14, there is a visual indication when the precise position of the locking device 37 is reached relative to the pedicle screw 1. In the same way, the locked position of the latching part 45 can be indicated visually with markings (not shown). During this process, in order to exert as good as no forces at all on the vertebral body 2 into which the corresponding pedicle screw 1 has been screwed, the handle 18 of the pliers 14 and the handle 53 of the sleeve 39 can be pressed against one another during the insertion of the locking device 37 and when locking.

As can be seen in FIG. 17 in particular, the rotating element 51 is provided with a continuous longitudinal borehole 55. A screwdriver 56 can be inserted into this borehole 55, as shown in FIGS. 14 to 16. With this screwdriver 56, the clamping bolt 44 (FIG. 17) in the locking device 37, which is attached to the pedicle screw and is in the locked position, can be rotated so that the insertion part 43 is pressed against the rod 24 that has been inserted into the pedicle screw. The locking device 37 is thus in the locked and tight position on the pedicle screw 1; the rod 24 is thus held optimally in the pedicle screw.

The means 38 for fitting and bracing of the locking device 37, guided by the second guide means 20 of the pliers and the fourth guide means 42 of the sleeve 39, are then drawn out of the pliers 14 that are still connected to the pedicle screw 1, and the arresting means 19 of the pliers 14 (FIGS. 4 and 5) can be released, and the pliers 14 can be removed from the pedicle screw 1.

In FIG. 18, the stabilization system for a vertebral column can be seen completed and fixed in place. The pedicle screws 1 are screwed into the vertebral bodies 2, the rod 24 that is inserted into the pedicle screws 1 is held optimally in the pedicle screws 1 by the locking devices 37, and both vertebral bodies 2 are therefore stabilized in optimal manner. Of course, to stabilize the vertebral bodies in the vertebral column, two stabilizing systems are fixed in place symmetrically.

The first guide means 11, the second guide means 20, the third guide means 31 and the fourth guide means 42 can be formed in known manner from ribs and grooves that fit together. Other guide means are of course also possible, for example guide rods and sleeves.

This instrument set of the invention enables accurate fixation of a stabilization system to the vertebral column of the body, all instruments used are accurately guided, the elements of the stabilization system that are to be fixed can therefore be brought very accurately to the correct position; an imprecise placement of these elements is practically ruled out. This feature is very attractive to a surgeon who carries out such an operation.

1. A instrument set for inserting a stabilization system, comprising anchoring elements, each having a screw holder and a U-shaped head part, into which head part a rod can be inserted for stabilization and be locked and latched by means of a locking device, into the spinal column of a body, comprising means for inserting the anchoring elements into the respective bone or vertebral bodies, means for inserting the stabilization rod into the inserted anchoring elements comprising pliers, and means for fitting and bracing the locking devices to the anchoring elements, wherein the means for inserting the anchoring elements have a head and a shaft, wherein the head is so constructed that it can be inserted into the U-shaped head part of the anchoring elements, wherein the shaft is equipped with first guide means, along which the pliers equipped with second guide means can be guided onto the head part of the anchoring element and coupled thereto, and wherein the means for insertion of the rod and the means for fitting and bracing the locking device are equipped with further guide means that interact with the second guide means of the pliers.

2. The instrument set according to claim 1, wherein the first guide means are attached to a guide sleeve, which guide sleeve can slide on the shaft.

3. The instrument set according to claim 1, wherein the pliers are provided with jaws, which grasp the outer sides of the U-shaped head part and which have cams, which engage with corresponding recesses in both branches, thereby coupling the pliers to the anchoring element.

4. The instrument set according to claim 3, wherein the jaws of the pliers are offset relative to the pivot axis of the pliers in the direction of the pivot axis.

5. The instrument set according to claim 1, wherein arresting means can lock the pliers in the position in which they are coupled to the anchoring element.

6. The instrument set according to claim 1, wherein the means for inserting the rod into the anchoring element are formed from a bar, on whose distal end a press-in part and on whose proximal end a handle is attached, and that the bar is provided with third guide means, which interact with the second guide means of the pliers.

7. The instrument set according to claim 1, wherein the means for fitting and bracing of the locking device are formed from a sleeve, on whose distal end a seating part is fixed to receive the locking device, and which sleeve is equipped with fourth guide means, which interact with the second guide means of the pliers.

8. The instrument set according to claim 1, wherein the locking device consists of an insertion part, a latching part and a clamping bolt.

9. The instrument set according to claim 8, wherein the seating part is provided with slot-like recesses, in which the latching part of the locking device can be rotated, and the locking device is thus held in the seating part.

10. The instrument set according to claim 8, wherein a rotating element is inserted into the sleeve, the distal end of the rotating element being equipped with projections which, in the inserted state of the locking device, protrude into the seating part over the latching part so that the latching part can be rotated relative to the insertion part by turning the rotating element.

11. The instrument set according to claim 7, wherein the sleeve has a handle on its proximal end.

12. The instrument set according to claim 10, wherein the rotating element has a handle attached to the proximal end that projects past the sleeve.

13. The instrument set according to claim 10, wherein the rotating element has a continuous longitudinal borehole, and wherein a screwdriver, with which the clamping bolt of the locking device can be rotated, can be inserted into the continuous longitudinal borehole.
